(19) United States
(12) Patent Application Publication
(10) Pub. No.: US 2023/0349008 A1
(43) Pub. Date: Nov. 2, 2023

(54) PAPER-BASED SAMPLE TESTING DEVICES AND METHODS THEREOF

(71) Applicant: CORNELL UNIVERSITY, Ithaca, NY (US)

(72) Inventors: Ruisheng WANG, Ithaca, NY (US); David ERICKSON, Ithaca, NY (US)

(21) Appl. No.: 18/025,965

(22) PCT Filed: Sep. 15, 2021
(86) PCT No.: PCT/US2021/050510

§ 371 Date: Mar. 13, 2023

Related U.S. Application Data
(60) Provisional application No. 63/078,377, filed on Sep. 15, 2020.

(51) Int. Cl. C12Q 1/70; C12Q 1/6844; B01L 3/00

(52) U.S. Cl. CPC C12Q 1/701; C12Q 1/6844; B01L 3/5023; B01L 2300/165; B01L 2200/16; B01L 2300/069; B01L 2300/126

(57) ABSTRACT

A sample testing chip includes a first layer formed of a porous hydrophilic material. One or more hydrophobic barriers are located in the first layer to define one or more testing areas configured to receive a volume of a sample and one or more auxiliary areas. The one or more testing areas and the one or more auxiliary areas are separated from one another by the hydrophobic barrier and are not fluidically connected. Methods of fabrication and use of the sample testing chip are also disclosed.

This application claims benefit of U.S. Provisional Patent Application Ser. No. 63/078,377 filed Sep. 15, 2020, the entirety of which is incorporated herein by reference.

This invention was made with government support under 1R01EB021331 awarded by National Institutes of Health. The government has certain rights in the invention.

### FIELD

The present technology relates to paper-based sample testing devices and more specifically to paper-based, semi-quantitative testing devices and methods thereof.

### BACKGROUND

In paper-microfluidics, 2D/3D channels are formed in a paper substrate through wax patterning to enable passive fluid transport. Compared to the polymers commonly used to fabricate microfluidic devices, paper substrates offer greater manufacturing flexibility through compatibility with a number of patterning techniques, act as a natural medium for colorimetric tests, and can be easily disposed of via incineration. Thus, paper-based microfluidics can be employed in a variety of applications.

Antimicrobial resistance (AMR) is increasingly recognized as a substantial threat to global health. Overuse and misuse of antimicrobials are contributing towards an increasing prevalence of antimicrobial resistant and multidrug resistant organisms. In the case of antibiotics, studies have shown that almost one in three prescriptions are inappropriate, either in choice of agent or duration. Incorrect antimicrobial usage can facilitate the development of antimicrobial resistance genes through selective pressures. In order to combat misuse, evidence-based antimicrobial agent selection based on antimicrobial susceptibility testing (AST) forms a key component of antimicrobial stewardship efforts. Accessible susceptibility testing methods are crucial for facilitating stewardship, especially in point-of-care and limited-resource settings. Clinically, the need for accessible testing methods remains unmet as many US hospitals outsource susceptibility testing to reference laboratories. The preference for user-friendly testing methods is reflected in the fact that when testing is done in-house, hospital labs predominately favor the use of gradient diffusion, which is relatively simple but can require subjective interpretation of results. Reference laboratories on the other hand predominately favor use of broth microdilution, which provides quantitative minimum inhibitory concentration (MIC) information but requires high operator input and availability of diagnostic instrumentation.

There have been ongoing research efforts to develop phenotypic AST methods. Phenotypic AST, which involves direct measuring of organism growth in the presence of an antibiotic, is differentiated from genotypic AST, which involves detecting resistance genes to infer susceptibility profiles. Developments in the area of microfluidic AST methods have attempted to improve upon conventional AST methods through automation and lower sample volume requirements. Examples of microfluidic AST approaches include a self-loading chip device, a pH-sensitive hydrogel sensor, agarose channels that enable morphology tracking, on-chip broth dilution, and nanoliter arrays. Despite these advances, microfluidic approaches often face limitations of manufacturing and readout complexity, which have served as barriers to scalability and widespread adoption.

Paper-microfluidics have the potential to address some of the scalability limitations of microfluidic AST. Examples of paper-microfluidic AST approaches include a paper-polydimethylsiloxane (PDMS) hybrid disk diffusion culture device, a paper-PDMS cell culture array, and a paper-based 13-lactamase test.

Additionally, The COVID-19 pandemic has had a profound effect on nearly all parts of our lives. While the severe lockdown strategies we have undertaken have saved millions of lives they have taken a great toll on our economies and societies. To return to normalcy, public health authorities need to be able to provide and maintain confidence that our institutions can be stood up without risking broadening the pandemic. There are numerous ways in which governments and health agencies are providing this confidence but two of the most important methods have proven to be upscaling diagnostic testing and widespread contact tracing for those who are infected.

As the pandemic has progressed, the first phase of this testing focused on increased local sampling of those who were symptomatic followed by traditional diagnostic testing at large scale centralized facilities. This required 3 days to a week for results to be returned. In the second phase an increased emphasis on point-of-care (PoC) testing, through platforms like the Cepheid Xpert Xpress SARS CoV-2 test, enable results to be returned quicker, but at a higher cost, and without the highly parallelized capabilities of the centralized facilities.

The next phase of this effort will require us to make a shift in diagnostic testing to large scale screening of widely distributed individuals. Given the large number of asymptomatic carriers, the reopening of the elements of the economy that have large numbers of individuals entering from all over the world at once (e.g. international airports, universities, large workplaces) will not be possible with traditional high-throughput techniques such as temperature taking, large-scale labs, and symptom reporting. In addition, this testing will need to be conducted in non-traditional settings—like pop-up clinics—without standard medical infrastructure. The existing PoC systems are largely serial in nature with relatively low throughput and therefore will not likely meet the large-scale screening need. What will be required here is a portable system that could process a much larger number of tests, much cheaper, and without the need for medical infrastructure. The system should also be able to seamlessly integrate with electronic platforms for contract tracing.’

A major challenge going forward with COVID-19 diagnostics is going to be (1) scaling up the throughput of point-of-care testing and (2) enabling it to be done in situations where traditional infrastructure may be limited, unreliable or non-existent and (3) combining it directly with contract tracing apps accessible to more quickly track others who may have become infected. There are several reasons for this, two of the most significant are as follows.

Clinical and epidemiological studies have demonstrated that there are a significant number of asymptomatic and pre-symptomatic carriers of the virus. The uncertainty in being able to detect these asymptomatic carriers using traditional means (such as forehead thermometers or questionnaires), coupled with the absence of widespread diagnostic screening means that the operation of large sections of our economy will have little confidence in being able to resume “normal pre-COVID” activities. International travel would be reliant on either massive quarantine restrictions or be limited in capacity as tests are processed serially. Universities will have limited capabilities to deliver on-campus educational services without being able to provide confidence to the students, staff and faculty of who may or may not be infected.

Moreover, in countries (or regions of countries) with limited access to advanced, large-scale clinical laboratory-based testing infrastructure, it is possible that cases are being underreported and the spread of the virus is broader than has been reported to date. In that case, there could be a very large number of carriers, symptomatic or otherwise, who, lacking an appropriate diagnosis, remain within the population continuing to spread the disease.

To addresses both these issues, there is a need to shift to a large-scale screening approach to detect cases—rather than the current diagnostic confirmation approach which follows the presentation of symptoms. To do this, the number of tests conducted will have to be increased dramatically and this testing will need to be distributed and likely deployed in non-traditional venues like temporary pop-up type clinics, within medical facilities that that do not traditionally have access to large-scale diagnostic testing (e.g. urgent care), and within areas or countries with limited resources. What is required for that is a portable system that could process a much larger number of tests, much cheaper, and without the need for medical infrastructure.

The present technology is directed to overcoming these and other deficiencies in the art.

### SUMMARY

One aspect of the present technology relates to a sample testing chip. The sample testing chip includes a first layer formed of a porous hydrophilic material. One or more hydrophobic barriers are located in the first layer to define one or more testing areas configured to receive a volume of a sample and one or more auxiliary areas. The one or more testing areas and the one or more auxiliary areas are separated from one another by the hydrophobic barrier and are not fluidically connected.

Another aspect of the present technology relates to a method for detecting a test target. The method includes providing a sample testing chip in accordance with the present technology. A test sample potentially comprising the test target is loaded to at least one of the testing areas. A control sample is optionally loaded to one or more control areas. The control sample is known as either comprising the test target or not comprising the test target. A supplementary liquid is loaded to at least one of the one or more auxiliary areas. A third layer is attached to a second surface of the first layer wherein no direct contact is formed between the third layer and either the one or more testing areas, the one or more auxiliary areas, or the one or more control areas. A volume is formed between the third layer and the first layer wherein the air layer is sealed. The sample testing chip is incubated under a desired temperature for a desired period of time. The one or more testing areas and optionally the one or more control areas are examined for a signal indicating the presence of the test target.

The present technology provides a user-friendly paper-based test that provides visual readout of test results, such as bacterial antibiotic susceptibility for example, in a semi-quantitative format. The present technology utilizes on-chip paper microfluidics to enable multiplexed testing of multiple test samples, such as antibiotic dilutions, with a single sample addition step, replicating the functionality of traditional broth-dilution-based susceptibility testing in a simplified format. The present technology provides several advantages including low sample volume requirement and lack of need for humidity control during incubation. Eliminating the requirement of humidity control increases incubation flexibility—meaning incubation can take place outside of a traditional laboratory incubator and in devices such as an oven or hotplate. The present technology may be employed, for example, in phenotypic antibiotic susceptibility testing, as well as DNA/RNA amplification on paper for detection of targets such as COVID-19.

### BRIEF DESCRIPTION OF THE DRAWINGS

FIGS. 1A and 1B are top views of one embodiment of a sample testing chip of the present technology.

FIG. 2 is a side view of a sample testing chip of the present technology.

FIGS. 3A and 3B are top views of an alternative embodiment of a sample testing chip of the present technology

FIGS. 4A and 4B illustrate alternative configurations for a sample testing chip of the present technology.

FIGS. 5A-5F illustrate an overview of the chip fabrication and antimicrobial susceptibility testing process.

FIGS. 6A-6D illustrate quantitative colorimetric analysis of on-chip bacterial grown results.

FIGS. 7A and 7B illustrate comparisons of on-chip and off-chip antimicrobial susceptibility testing results.

FIG. 8 illustrates an exemplary platform for on-chip screening for asymptomatic SARS-CoV-2 carriers.

FIGS. 9A-9E illustrate test results testing results for a set of nested LAMP primers for SAS-CoV-2 on nasopharyngeal swabs from 735 COVID-19 patients.

### DETAILED DESCRIPTION

The present technology relates to paper-based sample testing devices and more specifically to paper-based, semi-quantitative testing devices and methods thereof.

One aspect of the present technology relates to a sample testing chip. The sample testing chip includes a first layer formed of a porous hydrophilic material. One or more hydrophobic barriers are located in the first layer to define one or more testing areas configured to receive a volume of a sample and one or more auxiliary areas. The one or more testing areas and the one or more auxiliary areas are separated from one another by the hydrophobic barrier and are not fluidically connected.

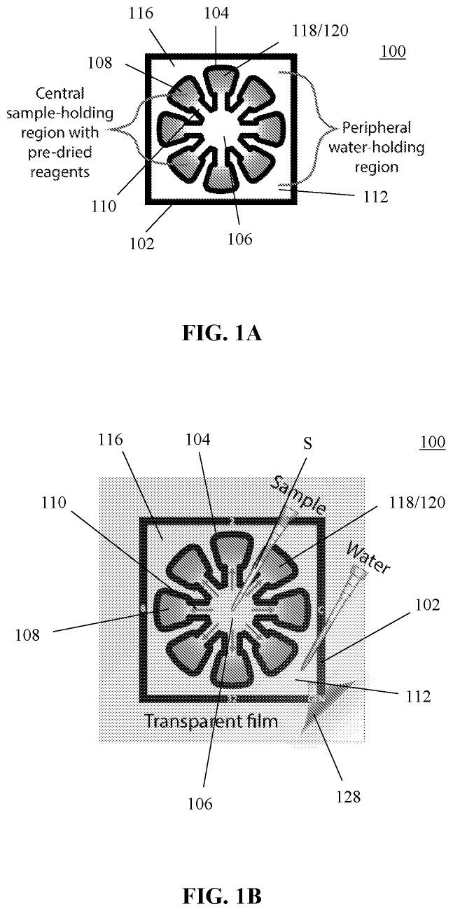

FIG. 1 is a top view of a first embodiment of a sample testing chip 100 of the present technology. Sample testing chip 100 may be utilized for example in phenotypic antibiotic susceptibility testing, as well as DNA/RNA amplification on paper for detection of targets such as COVID-19, although numerous other uses may be contemplated. The present technology provides a user-friendly testing device with a single sample addition step that requires low sample volume and lacks the need for humidity control during incubation.

Referring again to FIGS. 1A and 1B, sample testing chip 100 includes a first layer 102 and a hydrophobic barrier 104 located in the first layer 102. In this example, hydrophobic barrier 104 defines a central area 106, testing areas 108, channels 110, and an auxiliary area 112. In this example, hydrophobic barrier 104 defines eight testing areas 108 fluidically connected to central area 106 by channels 110, as well as auxiliary area 112 located around the peripheral edges of first layer 102, although sample testing chip 100 may include other numbers and configurations of hydrophobic barriers to provide other configurations including additional testing areas and auxiliary areas for example. It is to be understood that central area 106 and channels 110 are examples of one configuration that could be employed and are optional, i.e., sample testing chip 100 could include only testing areas and auxiliary areas. Additionally, sample testing chip 100 may include other types or numbers of layers or additional elements as described in further detail below. Non-limiting configurations include those described below with respect to FIGS.

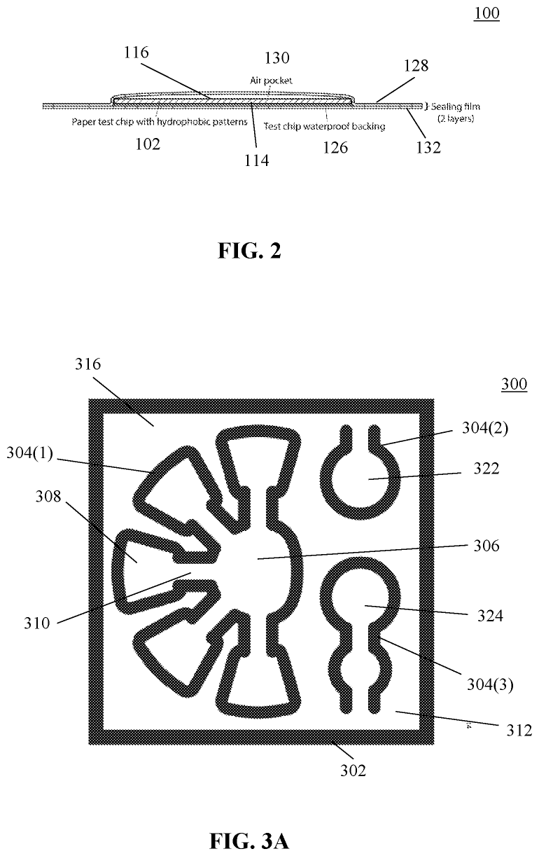

First layer 102 is formed of a porous hydrophilic material has a thickness that extends between a first surface (bottom surface) 114 to a second surface (top surface) 116, as shown in FIG. 2. The terms bottom and top surface are used merely to denote the position of first layer 102 during use and should not be construed as limiting. First layer 102 has a thickness between first surface 114 and second surface 116 in a range of 0.05 mm to 5 mm, in a range of 0.05 mm to 1 mm, or in a range of 0.1 mm to 0.5 mm, although other thicknesses may be employed. First layer 102 is formed of a material selected from the group consisting of paper, filter paper, chromatography paper, nitrocellulose, polyethersulfone (PES), cellulose-co-carbon fiber, cellulose-co-graphene, cellulose copolymer, polycarbonate, methylethyl cellulose, polyvinylidene fluoride (PVDF), polystyrene, glass, and any combinations thereof.

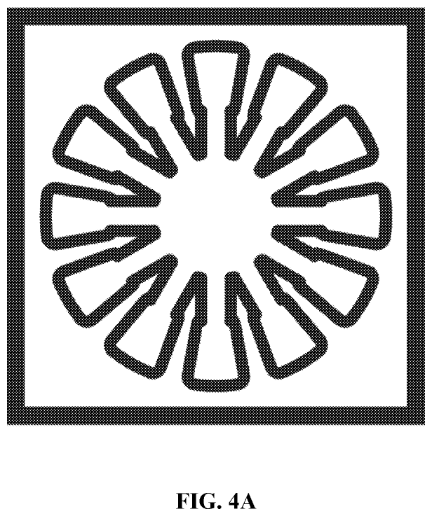

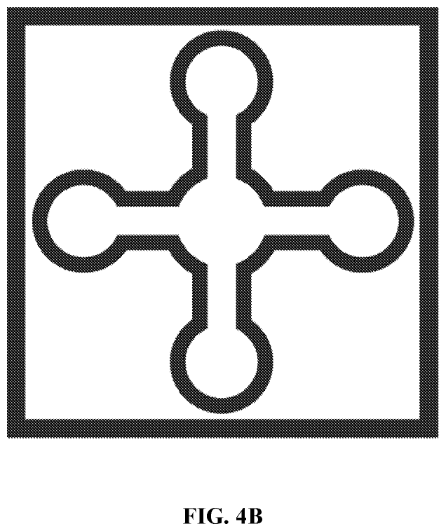

Referring again to FIGS. 1A and 1B, hydrophobic barrier 104 is located in first layer 102 and defines one or more areas in first layer 102, although additional hydrophobic barriers could be employed in other configurations to define other areas, such as control areas, within the first layer of a sample testing chip of the present technology as described in the examples herein, including FIGS. 4A and 4B as described below. Hydrophobic barriers could further be employed to form any number of testing areas or auxiliary areas without limitation. By way of example, FIGS. 4A and 4B illustrate alternative embodiments with twelve and four testing areas, respectively.

Referring again to FIGS. 1A and 1B, hydrophobic barrier 104 is formed by depositing or printing a hydrophobic substance in first layer 102 such that the hydrophobic substance extends along the thickness of first layer 102 between first surface 114 and second surface 116 (as shown in FIG. 2). The hydrophobic substance is formed of a material selected from the group consisting of wax, paraffin, ink, poly-methyl-methacrylate, polystyrene, polyvinyl chloride, polydimethylsiloxane (PDMS), silicone, acrylic acid-based polymers, methacrylic acid based polymers, acrylic acid-methacrylic acid based copolymers, and polyolefins, and modified polyolefins, and any combinations thereof.

Hydrophobic barriers are employed to define areas in the sample testing chips of the present technology that are separated and not fluidically connected to one another. By way of example only and without limitation, hydrophobic barriers may be shaped as lines, closed lines, an area, or a predetermined pattern. Referring again to FIGS. 1A and 1B, in this example, hydrophobic barrier 104 is patterned to define central area 106, testing areas 108, channels 110, and auxiliary area 112, although various configurations and numbers of hydrophobic barriers may be employed. Hydrophobic barrier 104 separates testing areas 108 from auxiliary area 112 such that testing areas 108 and auxiliary area 112 are not fluidically connected to one another, i.e., fluids introduced to testing areas 108 will not flow to auxiliary area 112 and vice versa. The size ratio between testing areas 108 and auxiliary area 120 may be in a range of 9:1 to 1:9, a range of 7:3 to 3:7, or in a range with an upper end of 9:1, 8:2, 7:3, 6:4, 5:5, 4:6, 3:7, or 2:8 and a lower end of 8:2, 7:3, 6:4, 5:5, 4:6, 3:7, 2:8, or 1:9.

In this example, testing areas 108 are each fluidically connected to central area 106 through channels 110. However, in other examples sample testing chip 100 could include any number of testing areas that are all fluidically separated from one another by hydrophobic barriers. In yet other examples, sample testing chip 100 could include a subset of testing areas that are fluidically connected to one another. For example, each of the testing areas could form a fluidic connection with at least 1, 2, 3, 4, 5, 6, 7, 8, 9, or 10 other testing areas. Further, although auxiliary area 112 is illustrated and described with respect to FIG. 1, it is to be understood that sample testing chip 100 could include other numbers of auxiliary areas that could form a fluidic connection with at least 1, 2, 3, 4, 5, 6, 7, 8, 9, or 10 other auxiliary areas on sample testing chip 100.

Referring again to FIGS. 1A and 1B, testing areas 108 are configured to receive a test sample (S). As illustrated in FIG. 1B, test sample (S), which may be a solution, a suspension, an emulsion, or a colloid, by way of example only, can be introduced into central area 106 and diffuse to each of test areas 108 by capillary action. In other examples, test sample (S) may be introduced directly to a testing area and diffuse to all other testing areas fluidically connected therewith. Sample testing chip 100 may be utilized with various test samples (S). Without limitation, the test sample (S) can be obtained from a plant, an animal, or a human. In one example, test sample (S) is from a patient. Testing areas 108 are configured to receive a volume of test sample (S) in a range of 1 tl to 1,000 tl, in a range of 10 tl to 500 tl, or in a range of 10 tl to 100 tl. The low sample volume requirement minimizes reagent consumption and is almost an order of magnitude lower than the per-well sample volume typically required in 96-well microdilution, as discussed in Wiegand, I., et al., “Agar and broth dilution methods to determine the minimal inhibitory concentration (MIC) of antimicrobial substances,” Nat. Protoc., 3, 163-175 (2008), the disclosure of which is incorporated by reference herein in its entirety

Sample testing chip 100 may be employed to receive test sample (S) that potentially includes a test target to be detected using sample testing chip 100. In one example, the test target, without limitation, can be a target microorganism that is naturally occurring or engineered, such as a bacteria, archaea, fungi, or any combinations thereof. More specifically, the test target can be a pathogenic microorganism. Test sample (S) can also include one or more nutrients suitable for growth of the target microorganism as known in the art. In another example, the test target is a target molecule that is naturally occurring or engineered, such as a gene, a deoxyribonucleic acid (DNA), a ribonucleic acid (RNA), an oligonucleotide, a polynucleotide, or any combinations thereof. More specifically, the target molecule in one example is a viral gene, such as the SARS-CoV-2 gene. In yet another example, the target molecule is from a pathogen, such as a virus. The virus can selected from the group consisting of a coronavirus, an influenza virus, a parainfluenza virus, a rhinovirus virus, an adenovirus, and any combinations thereof. It is to be understood that although example target microorganisms and molecules are described, sample testing chip 100 could be used with other microorganisms or molecules without limitation.

Referring again to FIGS. 1A and 1B, each of testing areas 108 includes a test reagent 118 located therein. In one example, as described in further detail below, test reagent 118 is dried into testing areas 108 of sample testing chip 100. Pre-drying of test reagent 118 simplifies the testing workflow and minimizes the number of operator steps needed to test, for example, multiple antibiotic concentrations. Testing areas 108 can include a volume of test reagent 118 in a range of 1 tl to 200 tl, in a range of 1 tl to 50 tl, or in a range of 1 tl to 10 tl, by way of example only. Testing reagent 118 is selected for detecting, for example, a target microorganism or a target molecule, as described above. Testing areas 108 can each include a different testing reagent to test for different targets.

In one example, testing reagent 118 further includes an indicator 120 that is capable of producing a chromogenic, colorimetric, fluorescent, luminescent signal, or any combinations of signals thereof at the presence of any of the target microorganisms described above. For example, test reagent 118 can include indicator 120 that is redox dye, such as resazurin, although other indicators suitable to produce the desired signals may be employed. In another example, sample testing chip 100 is utilized to measure antimicrobial resistance and test reagent 118 further includes an antimicrobial chemical, such as an antibiotic.

In another example, indicator 120 is capable of producing a chromogenic, colorimetric, fluorescent, luminescent signal, or any combinations of signals thereof at the presence of any of the target molecules described above. For example, test reagent 118 can include indicator 120 that is a pH indicator such as phenol red or bromothymol blue, for example. In another example, test reagent 118 further includes a reactive chemical suitable for a reaction with any of the target molecules describe above, for example. The reactive chemical can include a polymerase, a buffer, and a primer. The reactive chemical can also include a stabilizing agent selected from the group consisting of sucrose, bovine serum albumin (BSA), polyvinyl alcohol (PVA), and any combinations thereof. Indicator 120 and the reactive chemical can either be deposited in testing areas 108 simultaneously or at different times. Reactive chemical can, for example, be suitable to produce amplifying reaction and an optional reverse transcription in the presence of the target molecule. The amplifying reaction can include a conventional polymerase chain reaction (PCR) and/or an isothermal amplification wherein the isothermal amplification is selected from the group consisting of a loop-mediated isothermal amplification (LAMP), a strand displacement amplification (SDA), a helicase-dependent amplification (HDA), a recombinase polymerase amplification (RPA), a nucleic acid sequences based amplification (NASBA), a transcription mediated amplification (TMA), and any combinations thereof.

In any of the examples described herein, testing reagent 118 and indicator 120 may be deposited in testing areas 108 in the same concentrations, or in different concentrations throughout testing areas 108, depending on the desired testing.

Referring again to FIGS. 1A and 1B, auxiliary area 112 is located around the peripheral edge of first layer 102 based on the pattern of hydrophobic barrier 104. Auxiliary area 112 is fluidically separated from testing areas 108, as well as the optional central area 106 and channels 110. Auxiliary area 112 is configured to receive a volume of liquid, such as water, for example. In one example, as described in further detail below, auxiliary area 112 receives a liquid to provide humidity control from sample testing chip 100 during the sample testing process. Although a single auxiliary area 112 is illustrated and described with respect to FIGS. 1A and 1B, it is to be understood that additional hydrophobic barriers could be employed to define additional auxiliary areas that are either fluidically connected to one another or fluidically separated from one another by the hydrophobic barriers. In one example, at least one auxiliary area forms a fluidic connection with at least 1, 2, 3, 4, 5, 6, 7, 8, 9, or 10 other auxiliary areas.

Referring again to FIGS. 1A and 1B, auxiliary area 112 can include a supplementary liquid that diffuses to areas on sample testing chip 100 fluidically connected to auxiliary area 112. The supplementary liquid can be water, a saline, a buffer, a humectant, or any combinations thereof, by way of example only. Auxiliary area 112 is configured to receive a volume of the supplementary liquid in a range of 10 μl to 1,000 μl, or in a range of 50 μl to 500 μl. The supplementary liquid, in one example, saturates auxiliary area 112.

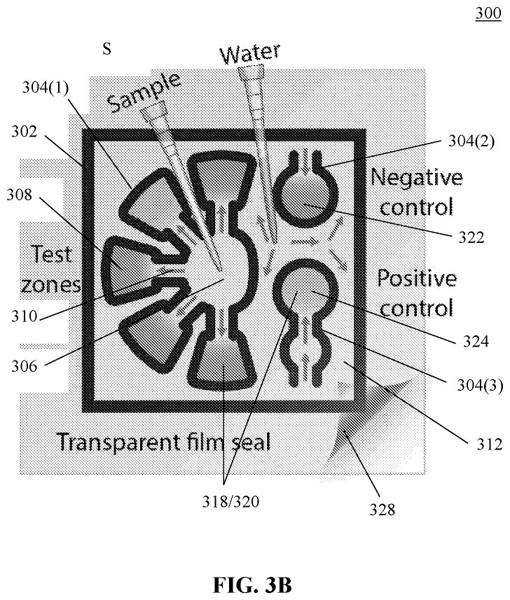

FIGS. 3A and 3B illustrate an alternative embodiment of a sample testing chip 300 of the present technology is illustrated. Sample testing chip 300 is the same in structure and operation as sample testing chip 100 except as described below. Sample testing chip 300 includes first layer 302. Sample testing chip 100 includes hydrophobic barrier 304(1) located in the first layer 302. In this example, hydrophobic barrier 304(1) defines a central area 306, testing areas 308, channels 310, and an auxiliary area 312. In this example, hydrophobic barrier 304(1) defines five testing areas 308 fluidically connected to central area 306 by channels 310, as well as auxiliary area 312 located around the peripheral edges of first layer 302.

Sample testing chip 200 also includes hydrophobic barriers 304(2) and 304(3) that define control areas 322 and 324, respectively. Control areas 322 and 324 are both fluidically separated from testing areas 308, as well as optional central area 306 and channels 310. Control areas 322 and 324 each form a fluid connection with auxiliary area 312 such that a supplementary liquid introduced into auxiliary area 112 will diffuse into control areas 322 and 324. In other examples, control areas 322 and 324 can be in fluid connection with at least 1, 2, 3, 4, 5, 6, 7, 8, 9, or 10 auxiliary areas. In yet another example, a sample testing chip of the present technology could include separate control areas that are fluidically separated from each other by a hydrophobic barrier.

Referring again to FIGS. 3A and 3B, control areas 322 and 324 are configured to receive a control sample that is known to either include or not include the test target. The test target can be any of the test targets described above, including target microorganisms and target molecules, for example. The control sample can be a solution, a suspension, an emulsion, or a colloid, by way of example. The control sample can further include one or more nutrients suitable for the growth of any of the target microorganisms described above.

Control areas 322 and 324 can further include test reagent 318, such as the test reagents described above. For example, test reagent 318 can be dried within control areas 322 and 324 as described in further detail below. Test reagent can further include an indicator 320 that includes any of the indicators described above, as well as any of the reactive chemicals described above.

Referring now more specifically to FIG. 2, the sample testing chip of the present technology, such as sample testing chip 100 can further include additional layers that interact with first layer 102, for example. Although the additional layers are described with respect to sample testing chip 100, those layers could be employed in the same manner respect to, for example, sample testing chip 300.

As illustrated in FIG. 2, sample testing chip 100 can further include second layer 126 coupled to first (bottom) surface 114 of first layer 102. Second layer 126 is in full contact with first surface 114 of first layer 102. Second layer 126 is formed of a water-proof material to prevent leakage from first layer 102. Second layer 126 is formed of a tape, a film, a plastic sheet, or a glass sheet, a rubber sheet, a silicone sheet, or any combinations thereof. Second layer has a thickness larger than 0.01 mm, in a range of 0.001 mm to 1 mm, or within a range of 0.01 mm to 0.2 mm, for example. Although second layer 126 is described as a single layer, it is to be understood that second layer 126 could include one or more sub-layers.

Sample testing chip can further include third layer 128 coupled to second (top) surface 116 of first layer 102. Third layer 128 is formed of a water-proof material to prevent leakage from first layer 102 when applied. Third layer 128 is formed from a tape, a film, a plastic sheet, or a glass sheet, a rubber sheet, a silicone sheet, a plastic cover, a glass cover, a rubber cover, a silicone cover, or any combinations thereof. Third layer 128 can be formed of a translucent or transparent material such that colorimetric changes in testing areas 108 can be visualized either by the human eye or imaging techniques, as described in further detail below. Third layer 128 has a thickness less than 5, 4, 3, 2, 1, 0.5 mm, in a range of 0.001 mm to 1 mm, or in a range of 0.01 mm to 0.2 mm. Although third layer 128 is described as a single layer, it is to be understood that third layer 128 could include one or more sub-layers.

In one example, third layer 128 is removable and/or reattachable to second surface 116 of first layer 102. When attached to second surface 116, third layer 128 does not directly contact testing areas 108 or auxiliary area 112 (third layer 128 similarly would not be in direct contact with control areas 322 and 324 as described with respect to FIGS. 3A and 3B). Third layer 128 forms a volume 130 located between third layer 128 and second surface 116 of first layer 102 that may encapsulate air, a gas, or vapor, for example. Volume 130 is sealed from the external environment and has a thickness less than 9, 8, 7, 6, 5, 4, 3, 2, or 1 mm. In another example, volume 130 has a thickness in a range of 0.001 mm to 3 mm, by way of example only.

As shown in FIG. 2, sample testing chip 100 can also include optional fourth layer 132 that is used to seal sample testing chip 100. Fourth layer 132 is coupled to second layer 126 as well as third layer 128 to seal sample testing chip 100 during use. Fourth layer 132 can be formed of the same material as described above with respect to third layer 128. Although fourth layer 132 is described as a single layer, it is to be understood that fourth layer 132 could include one or more sub-layers.

While exemplary sample testing chips 100 and 300 have been described, it is to be understood that various alterations could be made to the configuration of the sample testing chips including varying the number and shape of the testing areas, auxiliary areas, and control areas by varying the configuration of the hydrophobic barriers in the first layer.

Another aspect of the present technology relates to a method for detecting a test target. The method includes providing a sample testing chip in accordance with the present technology. A test sample potentially comprising the test target is loaded to at least one of the testing areas. A control sample is optionally loaded to one or more control areas. The control sample is known as either comprising the test target or not comprising the test target. A supplementary liquid is loaded to at least one of the one or more auxiliary areas. A third layer is attached to a second surface of the first layer wherein no direct contact is formed between the third layer and either the one or more testing areas, the one or more auxiliary areas, or the one or more control areas. A volume is formed between the third layer and the first layer wherein the air layer is sealed. The sample testing chip is incubated under a desired temperature for a desired period of time. The one or more testing areas and optionally the one or more control areas are examined for a signal indicating the presence of the test target.

An exemplary use of the present technology will now be described. First, sample testing chip 300 is provided, although the method may be utilized with other sample testing chips of the present technology, such as sample testing chip 100. Referring now to FIG. 3B, a method is described for analysis of a test sample (S) using sample testing chip 300.

First, test sample (S), which potentially includes the test target, is loaded to testing areas 308. Test sample (S) may be a solution, a suspension, an emulsion, or a colloid, by way of example only. Test sample (S), in this example, is introduced into central area 306 and diffuses to each of test areas 308 by capillary action. In other examples, test sample (S) may be introduced directly to a testing area and diffuse to all other testing areas fluidically connected therewith. Without limitation, the test sample (S) can be obtained from a plant, an animal, or a human. In one example, test sample (S) is from a patient. Test sample (S) is introduced in a volume in a range of 1 tl to 1,000 tl, in a range of 10 tl to 500 tl, or in a range of 10 tl to 100 tl.

Test sample (S) potentially includes a test target to be detected using sample testing chip 300. In one example, the test target, without limitation, can be a target microorganism that is naturally occurring or engineered, such as a bacteria, archaea, fungi, or any combinations thereof. More specifically, the test target can be a pathogenic microorganism. Test sample (S) can also include one or more nutrients suitable for growth of the target microorganism as known in the art. In another example, the test target is a target molecule that is naturally occurring or engineered, such as a gene, a deoxyribonucleic acid (DNA), a ribonucleic acid (RNA), an oligonucleotide, a polynucleotide, or any combinations thereof. More specifically, the target molecule in one example is a viral gene, such as the SARS-CoV-2 gene. In yet another example, the target molecule is from a pathogen, such as a virus. The virus can selected from the group consisting of a coronavirus, an influenza virus, a parainfluenza virus, a rhinovirus virus, an adenovirus, and any combinations thereof. It is to be understood that although example target microorganisms and molecules are described, sample testing chip 300 could be used with other microorganisms or molecules without limitation.

Testing areas 308 include testing reagent 318 suitable for detecting the test target, as well as indicator 320, which is capable of producing a chromogenic, colorimetric, fluorescent, luminescent signal, or any combinations of signals thereof at the presence of any of the target microorganisms described above. In one example, test reagent 318 is dried into testing areas 308 of sample testing chip 308. Testing areas 308 can include a volume of test reagent 318 in a range of 1 tl to 200 tl, in a range of 1 tl to 50 tl, or in a range of 1 tl to 10 tl, by way of example only. Testing reagent 318 is selected for detecting, for example, a target microorganism or a target molecule, as described above. In this example, each of testing areas 308 include testing reagent 318, although in other examples, at least one testing area can include the testing reagent, or each of the testing areas can include a different testing reagent to test for different targets.

Next, a control sample is optionally loaded to control areas 322 and 324. The control sample is known to either include or not include the test target. The test target can be any of the test targets described above, including target microorganisms and target molecules, for example. The control sample can be a solution, a suspension, an emulsion, or a colloid, by way of example. The control sample can further include one or more nutrients suitable for the growth of any of the target microorganisms described above. In this example, control area 322 is a negative control and does not include test reagent 318, while control area 324 is a positive control and includes test reagent 318 and indicator 320 dried therein.

Next, a supplementary liquid is loaded to auxiliary area 312 and diffuses to all areas fluidically connected including control areas 322 and 324. The supplementary liquid can be water, a saline, a buffer, a humectant, or any combinations thereof, by way of example only. Supplementary liquid can be added, for example, to provide for humidity control during incubation as described below. A volume of the supplementary liquid in a range of 10 μl to 1,000 μl, or in a range of 50 μl to 500 μl is introduced into auxiliary area 312. The supplementary liquid, in one example, saturates auxiliary area 312.

Next, third layer 328, such as a transparent film, is attached to first layer 302. Third layer 328 is formed of a water-proof material to prevent leakage from first layer 302 when applied. Third layer 328 is formed from a tape, a film, a plastic sheet, or a glass sheet, a rubber sheet, a silicone sheet, a plastic cover, a glass cover, a rubber cover, a silicone cover, or any combinations thereof. Third layer 328 can be formed of a translucent or transparent material such that colorimetric changes in testing areas 308 and/or control areas 322 and 324 can be visualized either by the human eye or imaging techniques, as described in further detail below. Third layer 328 has a thickness less than 5, 4, 3, 2, 1, 0.5 mm, in a range of 0.001 mm to 1 mm, or in a range of 0.01 mm to 0.2 mm. Although third layer 328 is described as a single layer, it is to be understood that third layer 328 could include one or more sub-layers. Further, it is to be understood that additional layers, such as described with respect to FIG. 2 could be employed during the method of the present technology.

Third layer 328 is attached such that it is not in direct contact with either testing areas 308, auxiliary area 312, or control areas 322 and 324 that can encapsulate air, a gas, or vapor, for example. The volume is sealed from the external environment and has a thickness less than 9, 8, 7, 6, 5, 4, 3, 2, or 1 mm. In another example, the volume has a thickness in a range of 0.001 mm to 3 mm, by way of example only.

Next, sample testing chip 300 is incubated under a desired temperature for a desired period of time. By way of example, the incubation period may be at least 1 minute, 10 minutes, 1 hour, 2 hours, 6 hours, 12 hours, 16 hours, 24 hours, 36 hours, or 48 hours. The temperature may be dependent on the test target and optimal growth or reaction conditions, by way of example.

After incubation, testing areas 308 and optionally control areas 322 and 324 are examined for a signal indicating the presence of the test target. By way of example, the signal may include a chromogenic, colorimetric, fluorescent, luminescent signal, or any combinations of signals thereof at the presence of any of the target microorganism or molecule. The use of a transparent film for third layer 328 allows for direct examination of sample testing chip 300. Sample testing chip 300 can be evaluated, for example, using the human eye. In other examples, sample testing chip 300 is examined using a device such as a camera, a scanner, a phone equipped with a camera, a stereoscope, a dissecting scope, or a microscope, or any combinations thereof to measure the signal produced.

### Example 1—On-Chip Antimicrobial Susceptibility Testing (AST)

### Example 1—Methods

Bacterial Strains

Quality control and multidrug-resistant reference strains with characterized AST profiles were sourced from the American Type Culture Collection (ATCC). Strains used in this study included Escherichia coli ATCC 25922, Escherichia coli BAA-2452, Klebsiella pneumoniae BAA-1903, and Acinetobacter baumannii BAA-1791. Strains were received in lyophilized format and revived according to vendor instructions.

Chip Design and Fabrication

2D microfluidic channels were designed using Adobe Illustrator and printed onto Whatman Grade 1 filter paper (GE Healthcare) using a ColorQube 8570 wax printer (Xerox). The channels were designed to separate the chip into two distinct regions: a central sample holding region that branches into eight test zones and a peripheral water holding region. Text labels were printed along the periphery of the chip to denote the antibiotic used and respective concentrations in each of the multiple zones. Following printing, each chip (5 cm length×5 cm width) was cut out using scissors sterilized with 70% ethanol. In order to melt the wax channels through the thickness of the filter paper to form a hydrophobic barrier, each chip was heated on a hot plate (VWR) set to 150° C. for one minute. After heating, the back side of each chip was sealed using sterile microplate sealing film (VWR) to prevent leakage. Alternatively, transparent tape can also be used.

To functionalize the chips for AST, antibiotics and the growth-sensitive dye resazurin were pre-dried on the chip to enable testing with a single sample addition step. Antibiotics used consisted of ampicillin, meropenem, gentamicin, and ciprofloxacin (all sourced from MilliporeSigma). The growth-sensitive dye resazurin (PrestoBlue, Thermo Fisher) is a redox indicator that is reduced by metabolically active cells into resorufin, leading to significant colorimetric and fluorescent changes which provide a visual indication of bacterial growth as disclosed in Guerin, et al., “Application of resazurin for estimating abundance of contaminant-degrading micro-organisms,” Lett. Appl. Microbiol., 32, 340-345 (2001), the disclosure of which is hereby incorporated by reference herein in its entirety. With 10× PrestoBlue solution as the diluent, antibiotic stock solutions prepared at 10 to 50 mg/ml (depending on solubility in water) were initially diluted to 2.5 to 10 mg/ml and subsequently two-fold serially diluted. When drying antibiotics on paper, it was necessary to use higher concentrations of antibiotics than that typically used in liquid cultures. 4 μl of each antibiotic-dye mixture was dispensed in the test zones in increasing concentration counterclockwise. A control zone without antibiotic was dispensed at the three o'clock position. Chips were dried at 37° C. for one hour and stored protected from light prior to use.

On-Chip AST

To prepare strains for on-chip AST, strains were first streaked on Mueller-Hinton II agar plates (Becton, Dickinson and Company) and incubated overnight at 37° C. in ambient air. Liquid cultures were then prepared by inoculating single colonies into Cation-Adjusted Mueller-Hinton Broth (CAMHB), followed by overnight incubation at 37° C. in ambient air. To achieve the recommended AST starting inoculum concentration of 5×105 CFU/mL²², the overnight culture was adjusted using CAMHB to the equivalent turbidity of a 0.5 McFarland standard, which represents a concentration of approximately 1×108 CFU/mL. Using a spectrophotometer (V-1200, VWR), the OD625 nm absorbance corresponding to a 0.5 McFarland standard was verified to be in the range of 0.08-0.13 22. Cultures were subsequently diluted 1:100 in CAMHB to reach an approximate starting concentration of 5×105 CFU/mL. 80 μl of diluted sample was then dispensed in the center of the test chip followed by 120 μl of distilled water in the peripheral water holding region. After the entire chip was saturated by the dispensed liquids, the chip was sealed using two pieces of sterile microplate sealing film (VWR). Sealing of the test chip prevents contamination and helps to maintain humidity by trapping evaporating water vapors. The water holding region increases the total volume of liquid stored on-chip, which helps to limit sample evaporation during incubation. Chips were incubated overnight (14-16 h) at 37° C. (ambient air) in a benchtop incubator protected from light.

Data Analysis

To quantitatively analyze color change in the test zones following incubation, test chips were imaged using a smartphone and analyzed in MATLAB (MathWorks). Images were analyzed in the hue, saturation, value (HSV) color-space which we determined to be less sensitive to lighting conditions compared to the red, blue, green (RGB) color-space. For each test zone, the average hue value in a 160-pixel diameter circular region was calculated and a threshold was used to differentiate between zones with reduced (growth) and unreduced (no growth) resazurin. The number of zones with reduced resazurin is correlated with the magnitude of the bacterial strain's MIC value.

Statistical Analysis

F-test was used to test for equality of population variances and unpaired one-tailed t-test was used to test for equality of population means.

### Example 1—Results

Paper-Based AST Design

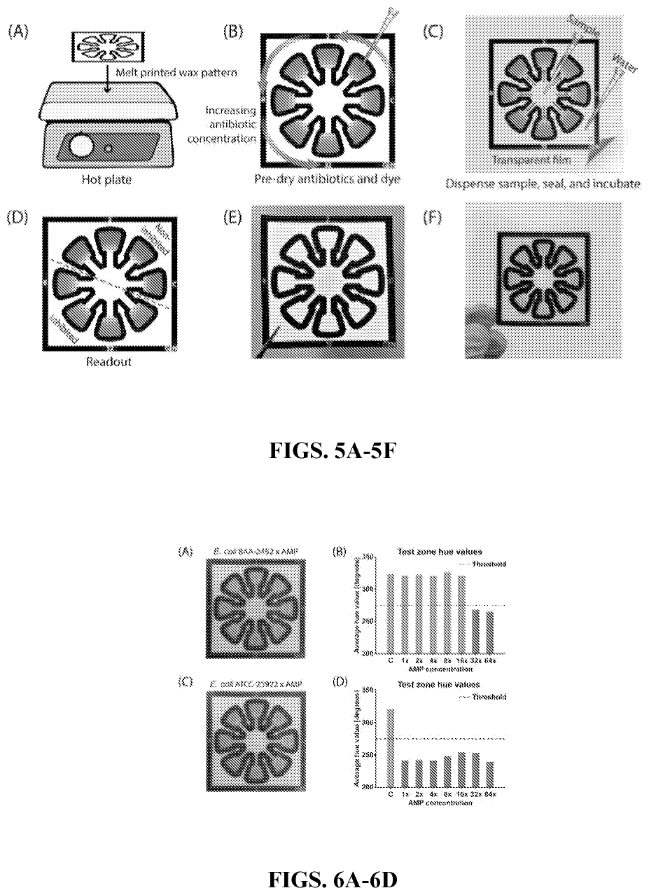

FIGS. 5A-5F illustrate an overview of the chip fabrication and AST process. A sealable paper-based test chip was developed that provides a visual readout of AST results. As shown in FIG. 5A, wax patterns printed on chromatography paper were heated on a hot plate to form hydrophobic barriers through the thickness of the papers. Hydrophobic wax channels were leveraged to create a network of test zones to enable susceptibility testing at multiple antibiotic concentrations with a single sample addition step. As shown in FIG. 5B, serial-diluted antibiotics (in this example gentamicin) along with the colorimetric redox indicator resazurin, which provides visual indication of bacterial growth, were pre-dried in the test zones, further simplifying testing workflow and replicating the functionality of broth-dilution-based AST. Each chip accommodates eight test zones that hold seven antibiotic concentrations along with a no-antibiotic control. Test zones increase in antibiotic concentration counterclockwise with the control zone at the three o'clock position. Prior to reagent dispensing, the back of the chip is sealed using sterile sealing film to prevent leakage. As shown in FIG. 5C, to initiate AST, bacterial sample is dispensed in the center of the chip and water is dispensed in the peripheral region. The chip is then sealed using sterile film to prevent contamination and trap evaporating water vapors, which helps maintain humidity during incubation. Increased fluid volume from the added water lowers sample evaporation during incubation. A single sample addition step reconstitutes dried reagents and allows bacteria to be incubated in the presence of a specific antibiotic concentration in each test zone. As shown in FIG. 5D, bacteria uninhibited by anitibiotics will reduce resazurin into resorufin, which induces a color change in the test zone. The redox indicator resazurin is reduced into resorufin by metabolically active bacteria as disclosed in Guerin, et al., “Application of resazurin for estimating abundance of contaminant-degrading micro-organisms,” Lett. Appl. Microbiol., 32, 340-345 (2001), the disclosure of which is incorporated by reference in its entirety, thereby providing a visual indication of bacterial growth when the test zone antibiotic concentration is insufficient to inhibit growth (i.e., below MIC). When considered collectively, the number of “positive” test zones exhibiting color change is correlated with the bacterial strain's MIC value and susceptibility category. FIG. 5E illustrates the test chip with melted wax channels and FIG. 5F shows the sealed test chip ready for incubation.

To initiate on-chip AST, 80 μl of sample is dispensed in the central region of the chip. The sample is then divided by the wax channels and flows into each of eight test zones via capillary action. Each test zone was estimated to accommodate 4 to 5 μl of sample volume. Due to these small sample volumes, a peripheral water-holding region was incorporated, which accommodates 120 μl of water, on the chip to minimize sample evaporation during incubation. Since the chip is sealed inside nonpermeable film during incubation, evaporating water vapors raise the ambient humidity of the internal air pocket and the increased fluid volume from the water-hold region helps mitigate sample evaporation.

Colorimetric Detection of Susceptibility

Following incubation, AST results can be interpreted qualitatively by eye or quantitively through colorimetric image analysis. Quantitative colorimetric analysis of on-chip bacterial grown results is shown in FIGS. 6A-6D. FIG. 6A illustrates a post-incubation test chip of E. coli BAA-2452 (AMP resistant via off-chip AST) grown in the presence of varying concentrations of ampicillin (AMP). Test zones with uninhibited and viable bacteria exhibit colorimetric change as resazurin is reduced into resorufin. The chip was imaged using a smartphone and analyzed in the hue, saturation, value (HSV) color-space. FIG. 6B illustrates the average hue value in a 160-pixel diameter circular region for each test zone and a threshold used to differentiate between zones with reduced (indicates growth) and unreduced (indicates no growth) resazurin. Zones above the threshold are classified as positive and zones below as negative. FIG. 6C illustrates a post-incubation test chip of E. coli ATCC-25922 (AMP susceptible via off-chip AST) grown in the presence of AMP. FIG. 6D shows test zone hue values of E. coli ATCC-25922. The chip was imaged using a smartphone and analyzed in the hue, saturation, value (HSV) color-space, which is more precise for colorimetric analysis and less sensitive to lighting conditions compared to the red, blue, green (RGB) color-space, as disclosed in Oncescu, V., et al., “Smartphone based health accessory for colorimetric detection of biomarkers in sweat and saliva” Lab Chip, 13, 3232-3238 (2013), the disclosure of which is incorporated by reference herein in its entirety. For each test zone, the average hue value in a 160-pixel diameter circular region was calculated and a threshold was used to differentiate between positive (uninhibited growth) and negative (inhibited growth) zones. A lower number of positive test zones correlates to a lower MIC value and a higher number of positive test zones to a higher MIC. As shown in FIGS. 6B & 6D, a significant difference in the hue profile can be used to distinguish between ampicillin-resistant (six positive zones) and ampicillin-susceptible (no-antibiotic control positive only) E. coli strains.

On-Chip AST

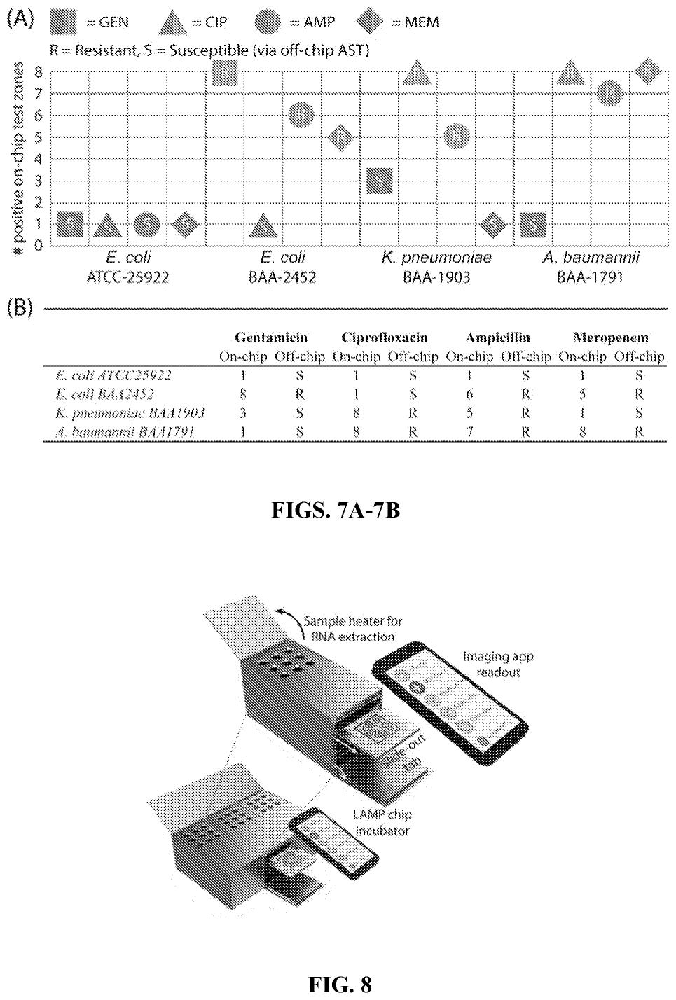

Using several clinically relevant bacterial organisms and antimicrobial agents, the colorimetric readout approach provides a strong predictor of susceptibility category. To demonstrate expanded on-chip AST capabilities, strains of E. coli ATCC-25922, E. coli BAA-2452, K pneumoniae BAA-1903, and A. baumannii BAA-1791 were tested against four common antibiotics: gentamicin, ampicillin, ciprofloxacin, and meropenem. Strains were tested at a recommended starting concentration of 5×105 CFU/mL as disclosed in Wiegand, I., et al., “Agar and broth dilution methods to determine the minimal inhibitory concentration (MIC) of antimicrobial substances,” Nat. Protoc., 3, 163-175 (2008), the disclosure of which is incorporated by reference herein in its entirety, and analyzed following overnight incubation. Each bacteria-antibiotic combination was tested in triplicate and the number of positive on-chip test zones, which showed consistency across triplicates for each combination, was correlated to off-chip AST determination. Comparisons of on-chip and off-chip AST results are shown in FIGS. 7A and 7B. FIG. 7A illustrates for each antibiotic, on-chip AST results in the form of number of test zones with reduced resazurin (indicates growth) are plotted along with off-chip AST determined susceptibility category. Square markers represent gentamicin (GEN), triangle markers represent ciprofloxacin (CIP), circle markers represent ampicillin (AMP), and diamond markers represent meropenem (MEM). R and S denote resistant and susceptible, respectively, as determined via off-chip AST. There is a statistically significant difference (P<0.001) in the number of positive test zones for susceptible and resistant categories. Each bacteria-antibiotic combination was tested in triplicate and the number of positive on-chip test zones showed consistency across triplicates for each combination. FIG. 7B illustrates a table summarizing AST results. For the on-chip results, the number indicates the number of test zones with reduced resazurin (including no-antibiotic control zone). For the off-chip results, S and R denote vendor MIC interpretations, corresponding to susceptible and resistant, respectively. The number of positive test zones is a strong predictor of susceptibility category.

The spatially separate test zones facilitate semi-quantitative interpretation of AST results, especially when aided by the resazurin-based colorimetric indicator. Imaging-based readout of results eliminates subjective interpretation and associated inaccuracies that can be present with visual-based readout of microdilution and gradient diffusion results, as discussed in Woods, G. L., et al., “Multisite Reproducibility of Etest for Susceptibility Testing of Mycobacterium abscessus, Mycobacterium chelonae, and Mycobacterium fortuitum,” J. Clin. Microbiol., 38,656-661 (2000) and Woods, G. L., et al., “Multisite Reproducibility of Results Obtained by the Broth Microdilution Method for Susceptibility Testing of Mycobacterium abscessus, Mycobacterium chelonae, and Mycobacterium fortuitum, J. Clin. Microbiol., 37, 1676-1682 (1999), the disclosures of which are incorporated by reference herein in their entireties. Compared to PDMS-based microfluidic devices, paper-based test chip of the present technology is of lower fabrication complexity. In addition, because sealing of the chip from the external environment removes the need for humidity control, the chip is amenable to flexible incubating conditions with temperature control as the only requirement—meaning incubation can occur in a benchtop incubator, oven, or even on a hotplate.

### Example 2—On-chip Screening for Asymptomatic SARS-CoV-2 Carriers

A modular and mobile phone enabled LAMP (Loop-mediated Isothermal AMPlification) based assay implemented on a paper-based chip format that is capable of meeting the cost, ease of use, and scaling potential required to meet the needs for large-scale screening as well as directly integrating with contact tracing software was developed. A core element of the modular Paper-COVID platform is an integrated sample processing system for both NP swaps and saliva samples.

As shown in FIG. 8, the first component of the platform includes a heating/incubator device to facilitate one-step RNA extraction and purification from patient samples. The second component of the platform includes a sealable paper-based test chip of the present technology for LAMP-based detection of viral nucleic acids. Paper-based test chips will be incubated inside a compartment of the heating device, which eliminates the need for a separate external incubator. The technology employs the SARS-CoV-2 LAMP assay disclosed in Wang, R., et al., “Rapid Diagnostic Platform for Colorimetric Differential Detection of Dengue and Chikungunya Viral Infections.” Analytical Chemistry, 91(8): 5415-5423 (2019), the disclosures of which are incorporated herein by reference in their entirety.

As shown in FIG. 8, the Paper-COVID system will be bifurcated into two compartments—a heating block compartment for micro-centrifuge tubes and a heat sink compartment that can incubate multiple paper-based test chips of the present technology. The heating block compartment will be capable of heating samples up to 95 degrees C., a temperature sufficient for viral inactivation, as disclosed in Bruce, E. A., et al., “DIRECT RT-qPCR DETECTION OF SARS-CoV-2 RNA FROM PATIENT NASOPHARYNGEAL SWABS WITHOUT AN RNA EXTRACTION STEP.” bioRxiv, 2020.03.20.001008 (2020), and Pastorino, B., et al., “Evaluation of heating and chemical protocols for inactivating SARS-CoV-2.” bioRxiv, 2020.04.11.036855 (2020), the disclosures of which are incorporated herein by reference in their entireties. The availability of commercial RNA extraction kits is currently a major bottleneck to large scale COVID-19 testing. The present technology is designed to primarily utilize heat to extract and purify RNA from patient samples. In the absence of commercial RNA extraction kits, heating is an alternative approach that can be leveraged to inactivate viruses and denature inhibitors/contaminants in the sample. A recent study demonstrated that even without the RNA extraction step, direct heating of nasopharyngeal patient samples followed by RT-qPCR can be used to inactivate RNAses and correctly detect positive samples with >90% accuracy, as disclosed in Bruce, E. A., et al., “DIRECT RT-qPCR DETECTION OF SARS-CoV-2 RNA FROM PATIENT NASOPHARYNGEAL SWABS WITHOUT AN RNA EXTRACTION STEP.” bioRxiv, 2020.03.20.001008 (2020), the disclosure of which is incorporated by reference herein in its entirety.

Following heat extraction, the sample will be dispensed and sealed in a paper-based LAMP test chip of the present technology. As shown in FIG. 8, a sealable paper-based test chip will enable downstream LAMP-based detection of viral nucleic acids following heat extraction. Stable wax channels will enable LAMP reagents, primers, and pH sensitive colorimetric dye to be pre-dried in different zones on the chip, as disclosed in Seok, Y., et al., “A Paper-Based Device for Performing Loop-Mediated Isothermal Amplification with Real-Time Simultaneous Detection of Multiple DNA Targets.” Theranostics, 7(8): 2220-2230 (2017), the disclosure of which is incorporated herein by reference in its entirety, thereby simplifying testing workflow. The presence of multiple test zones on a single chip may enable multiplexed detection of additional clinically relevant targets such as influenza, parainfluenza, rhinovirus, and adenovirus to facilitate differential diagnosis when non-specific patient symptoms are present.

A single sample dispensing step is needed to resolubilize reagents and initiate LAMP reactions within the test zones and a water dispensing step is needed for the spatially separate positive and negative control zones. The water dispensing step additionally serves to increase the humidity of the chip during incubation, which eliminates the need for external humidity control during incubation. Following the two dispensing steps, the chip is then sealed using a transparent film, which minimizes contamination as well as evaporation during incubation. Interpretation of test results is done colorimetrically and can be obtained qualitatively by eye or quantitatively by imaging with a camera or cell phone. With the result imaged on the cell phone the results can integrated with contract tracing apps and directly transmitted to local public health authorities.

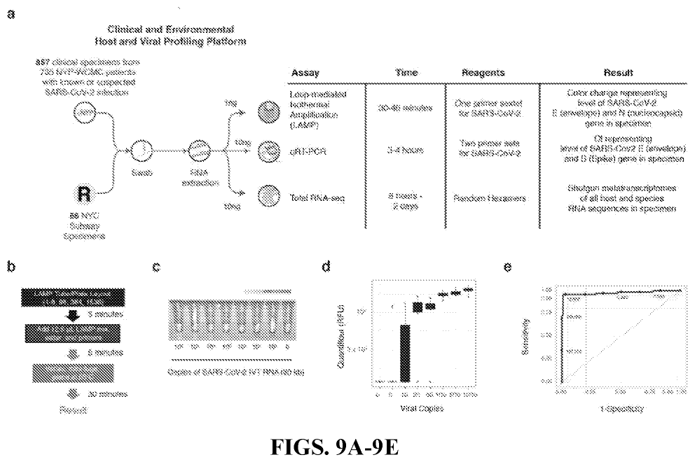

FIG. 9A illustrates test results for clinical and environmental samples collected with nasopharyngeal (NP) and isohelix swabs respectively, with RNA-sequencing, qRT-PCR, and LAMP. To gauge the clinical applicability of LAMP as a diagnostic assay and the need for a more rapid and simpler diagnostic test a set of nested LAMP primers for SAS-CoV-2 was developed and then tested on nasopharyngeal swabs from 735 COVID-19 patients (FIG. 9A), as discussed in Butler, D. J., et al., “Shotgun Transcriptome and Isothermal Profiling of SARS-CoV-2 Infection Reveals Unique Host Responses, Viral Diversification, and Drug Interactions.” bioRxiv, 2020.04.20.048066 (2020), the disclosure of which is incorporated by reference herein in its entirety. A set of six LAMP primers and paired workflow (FIG. 9B) for SARS-CoV-2 was first tested with a set of two synthetic RNAs from Twist Biosciences, based on the viral sequences of patients from Wuhan, China and Melbourne, Australia. The test samples were prepared using an optimized LAMP protocol from NEB, with a reaction time of 30 minutes. Primers were designed to create two nested loops and amplify within the SARS-CoV-2 nucleocapsid gene (N gene), which enabled a 30-minute reaction workflow. The first control (MT007544.1) was used to test the analytical sensitivity via the limit of detection (LoD), titrated from 1 million molecules of virus (106) down to a single copy, using serial log-10 dilutions. The reaction output was measured at 0-, 20-, and 30-minute intervals (FIG. 9C) before the samples were heated to 95 degrees C. for inactivation. The LoD was found to be between 10-100 viral copies per mL.

To further characterize reproducibility, sensitivity, and specificity for the LAMP assay, a range of experiments was performed. First, the SARS-CoV-2 RNA was serially titrated and measured with LAMP (FIG. 9D), as measured by QuantiFluor fluorescence, which showed decreasing fluorescence related to the total viral copies and an overlap of the median signal from negative controls at lower levels (5 total copies) of viral RNA (n=10). This translated to a 100% reproducibility at 1,000, 500, and 100 copies of viral RNA/mL, 95% reproducibility at 50 copies, and 90% reproducibility at 25 copies. This indicates an LoD threshold (95% reproducibility at two times the LoD) that is likely near 50 copies of RNA, with a maximum sensitivity of 5-50 copies of the viral RNA.

To further characterize the utility of the assay, a cohort of patient specimens was then tested to demonstrate the efficacy of the LAMP assay as a diagnostic approach on clinical specimens. The cohort consisted of 133 individuals that tested positive and 205 individuals that tested negative for SARS-CoV-2 by RT-PCR, of which 182 had enough material available for testing. The CDC-recommended, maximum cycle threshold (Ct) for diagnostic positives was 40, with an average of 23.1 Ct in the positive cohort and undetectable in the negative cohort. By using the fluorescence measurement of the previously (RT-PCR) established positive and negative samples as the gold standards, a Receiver Operator Characteristic (ROC) plot was then generated to estimate the diagnostic sensitivity and the specificity of the assay. After running the LAMP assay for 30 minutes, the resultant data showed an overall sensitivity of 96.4% and specificity of 99.7% (FIG. 9E). FIG. 9E illustrates the sensitivity and specificity of the LAMP assay from 201 patients (132 negative and 69 positive for SARS-CoV-2, as measured by qRT-PCR). Thresholds are DNA quantified by the QuantiFluor.

Although preferred embodiments have been depicted and described in detail herein, it will be apparent to those skilled in the relevant art that various modifications, additions, substitutions, and the like can be made without departing from the spirit of the application and these are therefore considered to be within the scope of the application as defined in the claims which follow.

1. A sample testing chip comprising:
a first layer formed of a porous hydrophilic material; and
one or more hydrophobic barriers located in the first layer to define one or more testing areas configured to receive a volume of a sample and one or more auxiliary areas, wherein the one or more testing areas and the one or more auxiliary areas are separated from one another by the hydrophobic barrier and are not fluidically connected.

2. The sample testing chip as set forth in claim 1, wherein the one or more auxiliary areas are configured to receive a volume of liquid to provide humidity control for the sample testing chip.

3. The sample testing chip as set forth in claim 1, wherein the size ratio between the one or more testing areas and the one or more auxiliary areas is in a range of 9:1 to 1:9.

4. The sample testing chip as set forth in claim 1, further comprising:
one or more additional hydrophobic barriers located in the first layer to define one or more control areas fluidically separated from the one or more testing areas, wherein the one or more control areas form a fluidic connection with at least one of said auxiliary areas.

5. The sample testing chip as set forth in claim 4, wherein the one or more control areas comprises at least two control areas that a fluidically separated from each other.

6. The sample testing chip as set forth in claim 1, wherein each of the one or more sample testing areas are fluidically separated from one another.

7. The sample testing chip as set forth in claim 1, wherein at least one of the one or more sample testing areas forms a fluidic connection with at least one other of said one or more sample testing areas.

8. The sample testing chip as set forth in claim 1, wherein at least one of the one or more auxiliary area forms a fluidic connection with at least one other of said one or more auxiliary areas.

9. (canceled)

10. The sample testing chip as set forth in claim 1 further comprising:
a second layer coupled to a first surface of the porous hydrophilic layer and/or a third layer coupled to a second surface of the porous hydrophilic layer.

11. The chip as set forth in claim 10, wherein the second layer is in full contact with the first surface of the porous hydrophilic layer.

12.-20. (canceled)

21. The sample testing chip as set forth in claim 10 further comprising:
a volume located between the third layer and the first layer configured to encapsulate air, a gas, or vapor, wherein the volume is sealed between the third layer and the first layer.

22.-28. (canceled)

29. The sample testing chip as set forth in claim 1, wherein the one or more testing areas are configured to receive a test sample potentially comprising a test target such that the test sample diffuses from said one or more testing areas to all other testing areas fluidically connected therewith.

30.-36. (canceled)

37. The sample testing chip as set forth in claim 29, wherein the test target is a target molecule wherein the target molecule is naturally occurring or engineered.

38. The sample testing chip as set forth in claim 37, wherein the target molecule is a gene, a deoxyribonucleic acid (DNA), a ribonucleic acid (RNA), an oligonucleotide, a polynucleotide, or any combinations thereof, and more specifically a viral gene, and even more specifically a SARS-CoV-2 gene.

39. The sample testing chip as set forth in claim 37, wherein the target molecule is from a pathogen, and more specifically from a virus, and even more specifically from a virus selected from the group consisting of a coronavirus, an influenza virus, a parainfluenza virus, a rhinovirus virus, an adenovirus, and any combinations thereof.

40. The sample testing chip as set forth in claim 1, wherein the one or more testing areas and one or more control areas further comprise a test reagent.

41.-51. (canceled)

52. The sample testing chip as set forth in claim 40, wherein the test reagent is suitable for detecting a target molecule wherein the target molecule is naturally occurring or engineered.

53. The sample testing chip as in claim 52, wherein the target molecule a gene, a deoxyribonucleic acid (DNA), a ribonucleic acid (RNA), an oligonucleotide, a polynucleotide, or any combinations thereof, and more specifically a viral gene, and even more specifically a SARS-CoV-2 gene.

54. The sample testing chip as set forth in claim 52, wherein the target molecule is from a pathogen, and more specifically from a virus, and even more specifically from a virus selected from the group consisting of a coronavirus, an influenza virus, a parainfluenza virus, a rhinovirus virus, an adenovirus, and any combinations thereof.

55.-67. (canceled)

68. A method for detecting a test target, comprising the following steps:
providing the sample testing chip of claim 4;
loading a test sample potentially comprising the test target to at least one of the testing areas;
optionally loading a control sample to the one or more control areas, wherein the control sample is known as either comprising the test target or not comprising the test target;
loading a supplementary liquid to at least one of the one or more auxiliary areas;
attaching a third layer to a second surface of the first layer wherein no direct contact is formed between the third layer and either the one or more testing areas, the one or more auxiliary areas, or the one or more control areas, whereby a volume is formed between the third layer and the first layer wherein the air layer is sealed;
incubating the sample testing chip under a desired temperature for a desired period of time; and
examining the one or more testing areas and optionally the one or more control areas for a signal indicating the presence of the test target.

69.-73. (canceled)
